(19) United States
(12) Patent Application Publication
(10) Pub. No.: US 2009/0324525 A1
(43) Pub. Date: Dec. 31, 2009

(54) FOUNDATION WITH HIGH CHROMATIC STABILITY AND METHOD TO OBTAIN SUCH FOUNDATION

(76) Inventor: Stefano GIANNI, Milano (IT)

Correspondence Address: Steinfl & Bruno, 301 N Lake Ave Ste 810, Pasadena, CA, 91101, US

(21) Appl. No.: 12/493,028

(22) Filed: Jun. 26, 2009

(30) Foreign Application Priority Data
Jun. 30, 2008 (EP) 08011781.5

(51) Int. Cl. A61K 8/18; A61Q 1/02

(52) U.S. Cl. 424/63

(57) ABSTRACT

Preparation of a foundation is described. The foundation is obtainable by mixing specific precursor emulsions, each of them having the complete composition of a foundation and containing a single pigment. The mixing directly provides the final foundation in the desired colour tone. Also described is a kit to obtain a foundation with improved stability, starting from the aforesaid emulsions.

### CROSS REFERENCE TO RELATED APPLICATIONS

The present application claims priority to European patent application EP 08011781.5, filed on Jun. 30, 2008 and incorporated herein by reference in its entirety.

### FIELD

The present disclosure relates to cosmetics and in particular to a foundation and related methods.

### BACKGROUND

Foundation is a cosmetic composition applicable on the face and other parts of the body.

Obtainment of a foundation with controllable and reproducible chromatic characteristics is desirable, for many reasons, including but not limited to faithfully reproducing the numerous natural nuances of the human skin.

Notwithstanding the wide research carried out in this sector, at the moment, the providing foundation compositions with controllable chromatic features and in particular, with high chromatic stability is still a challenge.

### SUMMARY

It has now been unexpectedly discovered that if foundation is prepared by mixing the necessary pigments not in their original state, but suitably separately pre-emulsified, it is possible to obtain foundation compositions with high chromatic stability.

The disclosure relates to a method for the preparation of a foundation which involves mixing suitable quantities of precursor monochromatic emulsions, each of them containing a single pigment, in which said mixing gives the final foundation in the desired colour tone. The present new preparation method is particularly useful for the cosmetic industry and the product distribution: in particular, the availability of an adequate stock of the mentioned precursor emulsions ensures industry the potentiality of producing foundation batches in almost infinite colour tones: at the same time, only the required colour tones are actually selected and produced in real time, in connection with market requirements and their variations.

In several embodiments, the process herein disclosed allows obtainment of a foundation composition with controllable chromatic features and in particular with high chromatic stability.

Additionally in several embodiments the process herein disclosed allows preparation of foundations with the above mentioned stability features in an easier manner if compared to certain methods known in the art.

Therefore, this production method allows optimising volumes and chromatic characteristics of the product in real time, avoids the creation and stocking of a material surplus and enables a better management of the orders and warehouse.

### DETAILED DESCRIPTION

The term “foundation” or “foundation composition” used herein refers to a cosmetic applicable the face or other parts of the body to create an even, uniform colour to the treated area and sometimes to change its natural skin tone and cover possible blemishes.

The term “emulsion” used herein refers to a system of a liquid dispersed with or without an emulsifier in an immiscible liquid usually in droplets of larger than colloidal size that can be used in cosmetic applications. Exemplary emulsions include homogeneous dispersions of the type water in oil (W/O) or oil in water (O/W), comprising the components used in cosmetic art, in particular water, a suitable oil like e.g. mineral oil, a surface active agent like e.g. lauric acid, linoleic acid, oleic acid, and derivatives thereof, as well as possible adjuvants like moistening agents, suspending agents, preservatives, active ingredients, perfumes, etc.

The term “means to measure” used herein refers generally to any means useful for ascertaining a property such as, quantity, size, weight, of a substance compared to a designated standard. Accordingly means to measure precursor emulsions indicate any device that can be used to determine suitable amounts of one or more precursor emulsion to be mixed for obtaining a desired colour tone according to the present disclosure. Exemplary means to measure include but are not limited to tubes, vials, beakers, flasks, and other devices identifiable by a skilled person upon reading of the present disclosure. Devices suitable as means to measure can possibly comprise marks suitable to measure the amount of fluid contained, or having a pre-set volume to be filled, corresponding to the amount of emulsion required.

The term “means to mix” used herein refers with generally to any means useful for combining components of a composition into one mass. Accordingly, means to mix precursor emulsions include any device that can be used to combine one or more precursor emulsions to provide a foundation according to the present disclosure. Exemplary means to mix include but are not limited to manual or electric stirrers, vibrating systems, spoons, spatulae, and other devices identifiable by a skilled person upon reading of the present disclosure.

The term “pigment” used herein identifies a substance used especially to impart color to a material and in particular to a cosmetic material. Pigments include those coloured substances, usually in a powder form compatible with the human skin, that are used in the cosmetic art. The term “mono-pigmented” refers to the property of containing only one pigment as above defined. The term “precursor emulsion” used herein refers to an emulsion as above defined, containing only one pigment.

Foundation is a cosmetic composition applicable on the face and other parts of the body, useful for giving skin a pre-determined colour and/or covering possible blemishes. Foundations are generally provided as emulsified creams, containing different pigments, duly mixed, so to reproduce the natural colour of the skin. Their related compositions contain lipophilic phases, for example silicones, emulsified with water by means of special surfactants.

In this field, it is particularly important to obtain compositions in a wide range of colours, whose chromatic characteristics can be easily controlled and reproduced, and having high stability characteristics in emulsified form. Typically, in the production of foundations, it is required to obtain a very high number of compositions in a very limited chromatic range, in order to reproduce the numerous natural nuances of the human skin in the most faithful manner. Within this limited chromatic range, colour differences between one composition and another are very small: hence, it is necessary to obtain highly stable compositions, reproducible as for final colour.

Normally, the preparation of the foundation envisages the creation of a primary emulsion, to which the mixture of pigments required for obtaining the desired colour tone is added; in other cases, pigments can be emulsified directly with the other ingredients, obtaining the final foundation, in a single step; in all these techniques, the final colour of the foundation is determined on the basis of qualitative-quantitative formulations of different pigments, which are used as such (i.e. as they are available on the market, for example in powder form) together with the other ingredients of the foundation. These techniques have some limits, especially with respect to the definition of the final colour obtained, which is not always optimal. In fact, it has been noticed that, starting from identical pigment formulations and subjecting them to the same formulation process, foundations were obtained with significant colour variations among different batches or even within one single production batch.

Recently, in U.S. Pat. No. 6,284,228 (incorporated herein by reference in its entirety), it has been proposed to create a foundation by mixing different, separately prepared components, respectively: one light component, one red component, one green component and one optional dark component; the colour of the foundation results from mixing of the abovementioned components; in turn, each component contains mixtures of different pigments, to be mixed with one another as such, in specific percentages: therefore, this technique shows the same limits of the original technique, i.e. the variability of the final colour, since it is predetermined starting from mixtures of pigments as such.

According to the present disclosure, the final colour of the foundation is not determined by mixing of pure pigments (i.e. as such as they are available on the market, for example in powder form); in this disclosure, each pigment is first separately emulsified, thus obtaining a corresponding number of precursor emulsions: mixing the precursor emulsions in predetermined quantities leads to the obtainment of the foundation in the desired colour tone. Therefore, one of the characteristics of this disclosure lies in that each precursor emulsion is not simply monochromatic upon observation, but also mono-pigmented: its colour is not the result of different pre-mixed pigments, but of the use of one single pigment; consequently, every contact among pigments occurs exclusively by mixing emulsions separately containing them: said mixing leads to the desired final colour tone of the foundation.

In a further embodiment of the disclosure, each precursor emulsion already has the complete composition of a normal foundation, differing from those known in that it contains one single pigment (red, yellow, black, etc.): in this case, the final foundation can be obtained directly by merely mixing the single precursor emulsions; these emulsions, having an identical composition, are perfectly miscible with one another and lead to foundations with high homogeneity, being quickly obtainable, with limited energy consumption, with no need of further ingredients and/or treatments.

The high miscibility of the precursor emulsions has very important implications: at the industrial level, for example, the preparation time was found to be substantially halved, compared to the traditional technique, as verified by tests carried out by the Applicant; in addition, the requirement of mixers with high energy consumption (turbo-mixers) is avoided, since traditional mixers can be used, quickly obtaining a product with high homogeneity. At the laboratory level, the high miscibility allows quickly obtaining new experimental colour tones in a reliable and reproducible manner.

In general, on any preparation scale, mixing these precursor mono-pigmented emulsions proved to be easier and immediate compared to the traditional mixing of basic ingredients (heterogeneous phases, such as powder pigments, lipophilic phases, hydrophilic phases, various additives, etc.). For the same reasons, it is extremely easy and immediate, both on an industrial and laboratory scale, to slightly adjust or alter the final colour by adding small portions of a mono-pigmented emulsion, whose formulation is equal to the one to be adjusted: these operations are much longer and more difficult if the adjustment occurs by adding pigments as such to the foundation, as known in the art. The finding that the production method is directly implementable on the common mixers used to produce the traditional foundations, with no need to adjust the plant, is a further useful aspect of the present disclosure.

The aforesaid preparation method proved to be useful also from the colour stability and reproducibility standpoint, enabling the obtainment of foundations high stability both within a single batch and among different batches. By stability within a batch, it is meant the colour constancy of foundation aliquots belonging to the same production batch, though obtained at different times. By stability among different batches, it is meant the colour constancy of foundations belonging to different batches, though obtained from the same original formulation.

The aforementioned effects are particularly amplified when the precursor emulsions are silicone-based emulsions (water in silicone) and the pigments used are of the types selected by the Applicant. Examples of silicone raw materials, useful for the preparation of the silicon-based emulsions are: Methicone and Dimethicone, Phenyl Trimethicone, Cyclopentasyloxane.

The pigments selected by the Applicant according to one useful embodiment of the present disclosure are silicon-treated pigments, using silicones such as Methicone, Dimethicone or others. For information purposes, among the pigments available on the market, those of the FDP series (Toshiki Pigment), AS series (ex. Wacker colouring agents), SA series (ex. Miyoshi Kasei), etc., can be mentioned.

Precursor emulsions are used in a limited number, for example four; the colours of the single pigments contained therein, can be: black, red, yellow and white; the corresponding pigments can be: black iron oxide, red iron oxide, yellow iron oxide and titanium dioxide.

The pigment concentration within each emulsion and the quantities of emulsions to be mixed can widely vary depending on the final desired colour. Emulsions are prepared and combined with one another in such quantities so to obtain the qualitative-quantitative ratio of pigments necessary for achieving the desired target colour: the ratios between pigments in question, per se known in cosmetology, are herein applied to the corresponding mono-pigmented emulsions, obtaining the abovementioned benefits.

The remaining ingredients of the precursor emulsions are raw materials for cosmetic use, also commonly intended for the preparation of foundations. By way of a non-limiting example, we can mention: dispersants, moistening agents, emulsifying agents, suspending agents, preservatives, active ingredients, perfumes, etc.

Even the preparation methodology of the single emulsions can be chosen among those commonly known standards.

The fractioning of the foundation in the present precursor emulsions, allows also the preparation of the “on the spot” foundation. By “on the spot” preparation, we mean the preparation carried out only in case of real necessity: this preparation is, for example, that carried out on a personal scale by the end user, at the time of application on the skin; an “on the spot” preparation is also that made on the intermediate scale by the wholesaler or by the pharmacist, just before selling it; an “on the spot” preparation includes also the preparation carried out by the cosmetic industry itself, which can produce precursor emulsions as main finished product, reserving the production of final foundations only in targeted quantities and at the time of a real necessity, for example in response to a specific order. Such production method allows a significant modification of the whole production chain, making it substantially targeted to the obtainment/stocking/transport of a very limited number of precursor emulsions (for example four), from which targeted productions of specific foundations are obtained. This methodology represents a basic difference compared to known productions, which aim from the start to obtain final foundations in innumerable colour tones: this methodology involved a complicate management of the warehouse and distribution, also exposing the manufacturer to the store of unsold foundations (typically in the least-used colours), or obliging him to separately produce and stock innumerable foundation colours, in different quantities depending on the estimated sale volume for each colour tone.

On the other hand, according to the disclosure, precursor emulsions are sold to the wholesaler, pharmacist or end user, while leaving these subjects the task of producing the final foundation at the desired moment; this preparation can be carried out with the aid of special instructions (especially those for obtaining predetermined colour nuances) and possible means for easily performing the mixing.

Therefore, the disclosure includes also a kit for the preparation of a foundation including a specific number of mono-pigmented precursor emulsions, each emulsion having, according to some embodiments, the complete composition of a foundation and the following optional components: one or more containers to mix emulsions, one or more means to meter them, one or more means to mix them and instructions which indicate the quantities of emulsions to be used for each colour nuance.

### Example 1

By way of a non-limiting example of the present disclosure, four precursor emulsions were prepared with the same formulation base, made up of the following ingredients: water, cyclopentasiloxane, talc, mineral oil, glycerine, propylene glycol, Cetyl PEG/PPG-10/1, Dimethicone, Polyglyceril-3 diisostearate, sodium chloride, nylon 12, disteardimonium hectorite, dimethicone/vinyl dimethicone crosspolymer pigment, dimethicone/methicone copolymer pigment, tocopheryl acetate, sodium dehydroacetate, EDTA tetrasodium, phenoxyethanol, ethylhexylglycerin.

The only difference among the four emulsions consisted in the single pigment contained therein, that is titanium dioxide, yellow iron oxide, red iron oxide and black iron oxide, respectively. The precursor emulsions were then mixed with one another in such quantities as to obtain a final foundation emulsion containing:

| Pigment | % Quantity (of the pigment total) |
|---|---|
| Titanium dioxide | 44.7% |
| Yellow iron oxide | 21.5% |
| Red iron oxide | 19.7% |
| Black iron oxide | 14.1% |

Mixing was carried out in a mixer of Melter/Mixer type for pastes and cosmetic compounds, carried out at a speed of 45 rpm, for a total time of 20 minutes.

A silicone foundation of Beige No. 1 colour, with high homogeneity, was obtained. Repeated tests have highlighted a high colour stability and reproducibility, both within the same production batch and between different batches. In addition, the product has passed the following safety tests envisaged for placing cosmetic products on the market: stability test (stability of the formula over time), challenge test (effectiveness of the preservative system), patch test (skin irritation test).

Accordingly, what has been shown are a foundation with high chromatic stability and a method for obtaining such foundation. While such foundation and method have been described by means of specific embodiments and applications thereof, it is understood that numerous modifications and variations could be made thereto by those skilled in the art without departing from the spirit and scope of the disclosure. It is therefore to be understood that within the scope of the claims, the disclosure may be practiced otherwise than as specifically described herein.

The examples set forth above are provided to give those of ordinary skill in the art a complete disclosure and description of how to make and use the embodiments of the compositions and methods of the disclosure, and are not intended to limit the scope of what the inventors regard as their disclosure. All patents and publications mentioned in the disclosure are indicative of the levels of skill of those skilled in the art to which the disclosure pertains.

The entire disclosure of each document cited (including patents, patent applications, journal articles, abstracts, laboratory manuals, books, or other disclosures) is hereby incorporated herein by reference to the same extent as if each reference had been incorporated by reference in its entirety individually.

It is to be understood that the disclosures are not limited to particular compositions or systems, which can, of course, vary. It is also to be understood that the terminology used herein is for the purpose of describing particular embodiments only, and is not intended to be limiting. As used in this specification and the appended claims, the singular forms “a,” “an,” and “the” include plural referents unless the content clearly dictates otherwise. The term “plurality” includes two or more referents unless the content clearly dictates otherwise. Unless defined otherwise, all technical and scientific terms used herein have the same meaning as commonly understood by one of ordinary skill in the art to which the disclosure pertains.

Specific materials and methods are described herein as examples of appropriate materials and methods suitable for obtaining the compositions and process herein described. Accordingly, any methods and materials similar or equivalent to those described herein can be used for obtaining the composition and process of this disclosure.

A number of embodiments of the disclosure have been described. Nevertheless, it will be understood that various modifications may be made without departing from the spirit and scope of the present disclosure. In particular, modifications of the above-described modes for carrying out the disclosure that are obvious to persons of skill in the art are intended to be within the scope of the present disclosure. Accordingly, various embodiments are within the scope of the following claims.

1. Process for preparation of a foundation, the process comprising
mixing quantities of a plurality of precursor emulsions, each precursor emulsion containing one single pigment, wherein the mixing provides the foundation with a desired colour tone.

2. The process according to claim 1, wherein each precursor emulsion contains a complete composition of a foundation, each precursor emulsion differing from another only in the pigment contained therein.

3. The process according to claim 1, wherein the precursor emulsions are four in number, having respectively a black, red, yellow and a white colour.

4. The process according to claim 3, wherein the pigments are: black iron oxide, red iron oxide, yellow iron oxide and titanium dioxide, respectively.

5. The process according to claim 1, wherein the pigments are silicone-treated pigments.

6. The process according to claim 1, wherein the precursor emulsions are water in silicone emulsions.

7. The process according to claim 1, wherein the process is carried out on an industrial scale, the process further comprising
providing users with an already mixed final product.

8. The process according to claim 1, wherein the process is individually carried out by users, and wherein the mixing is performed by
selecting a desired colour tone and
mixing the precursor emulsions according to proportion indications to obtain the desired colour tone.

9. A precursor emulsion containing a single pigment together with remaining ingredients necessary to form a complete composition of a ready-to-use foundation.

10. The precursor emulsion according to claim 9, wherein the single pigment is selected from the group consisting of: black iron oxide, red iron oxide, yellow iron oxide and titanium dioxide.

11. The precursor emulsion according to claim 9, wherein the pigment is a silicone-treated pigment.

12. The precursor emulsion according to claim 9, wherein the emulsion is a water-in-silicone emulsion.

13. Kit for preparation of a foundation including a plurality of precursor emulsions according to claim 9.

14. The kit according to claim 13, further comprising at least one of the following components: one or more containers to mix the precursor emulsions, one or more measuring means to measure the precursor emulsions, one or more mixing means to mix the precursor emulsions and instructions indicating quantity of emulsion to be used for each colour tone.

15. The kit according to claim 13, wherein the quantities of emulsions contained in the kit are selected to allow preparation of a single foundation package for personal use.

16. The kit according to claim 13, wherein the quantities of emulsions contained in the kit are selected to allow preparation of a quantity of foundation consistent with requirements of a wholesaler, drugstore or another intermediate distributor.
